(19) United States
(12) Patent Application Publication
(10) Pub. No.: US 2018/0085008 A1
(43) Pub. Date: Mar. 29, 2018

(54) Health Metric Validation System

(71) Applicants: David R. Hall, Provo, UT (US); Dan Allen, Springville, UT (US); Min Kang, Provo, UT (US); Ben Swenson, Lehi, UT (US); Terrece Pearman, Draper, UT (US)

(72) Inventors: David R. Hall, Provo, UT (US); Dan Allen, Springville, UT (US); Min Kang, Provo, UT (US); Ben Swenson, Lehi, UT (US); Terrece Pearman, Draper, UT (US)

(21) Appl. No.: 15/279,734

(22) Filed: Sep. 29, 2016

(51) Int. Cl. A61B 5/0205; A61B 5/0402; A61B 5/145; A61B 5/00; G01N 33/493; G01N 33/483; G01N 33/49

(52) U.S. Cl. CPC A61B 5/0205; A61B 5/0402; A61B 5/14546; A61B 5/14535; A61B 5/021; A61B 5/7221; G01N 33/493; G01N 33/4833; G01N 33/49; A61B 5/0006

(57) ABSTRACT

We disclose a health metric validation system in which a first heath metric is taken which is a measurement that is relevant to a user's health status. The first health metric may be used to diagnose a disease. A second metric is collected from the user and used to validate the first health metric. The first health metric and the second metric are entered into a computer which applies a first set of rules to the first health metric and second metric. The computer calculates a weight value and assigns it to the first health metric. The computer applies a second set of rules to the first health metric and its weight value to calculate an indicator value. The indicator value provides an indication of the validity of the first health metric.

### BACKGROUND

### Field of the Invention

This invention relates to systems for determining health conditions.

### Background of the Invention

Every method of measuring physiological functions has inherent limitations. Medical devices and laboratory assays may provide inaccurate results for various reasons including user error, damaged components, or attempts to use the device or assay under conditions for which it was not designed. There are also circumstances under which the medical device or laboratory assay provides data that may not be properly interpreted without knowing specific information about the user which puts the data in proper context. Additionally, health care providers sometimes simultaneously use multiple health data inference methods, each associated with a different degree of accuracy and relevance, in an attempt to create a complex assessment of an individual's health or to select a single diagnosis out of a lengthy differential diagnosis. Each measurement may have a different shortcoming that must be taken into account when interpreting the data generated by the measurement. A way to determine the level of accuracy of health related data and to put the data in proper context to make it most meaningful is needed.

### BRIEF SUMMARY OF THE INVENTION

We disclose a novel system for identifying the level of validity of health metrics. This system may also be used to assess the best context in which to interpret health metrics by identifying the body type and/or other relevant physical characteristics. This system comprises the collection of a first metric which is relevant to the user's health status. A second metric is collected which is an indicator of the validity of the first metric. The first and second metrics are analyzed according to a first set of rules which assign a weight value to the first metric. A second set of rules calculates an indicator value for the first metric, the indicator value being a function of the weight value.

The first and second set of rules may vary depending on physiological characteristics, including, but not limited to body type, gender, skeletal structure (fine or heavy) and whether or not the user is afflicted with a certain disease. A healthcare provider may enter information about the user's specific physiological characteristics into the computer to trigger the alternative set of rules. Alternatively, the system may trigger the collection of a follow-up metric which may determine whether the user has a physiological characteristic that may then trigger the application of an alternate set of rules to calculate and/or interpret the first metric.

In some embodiments of the invention, the first and/or second metrics are conducted by a medical toilet. Some embodiments of the medical toilet may then transmit the metrics electronically to a computer programmed to analyze the data for validity. The system may then signal the medical toilet to conduct a follow-up metric as described herein.

### BRIEF DESCRIPTION OF THE DRAWINGS

FIG. 1 is a perspective view of one embodiment of the system in which a first and second metric are collected and entered into a computer.

FIG. 2 is perspective view of one embodiment of the system in which the first metric is an electrocardiogram (EKG) reading and the second metric is a measurement conducted by a medical toilet.

FIG. 3 is a flow chart illustrating an embodiment of a decision making process for assessing the validity of a first metric.

FIG. 4 is a perspective view of one embodiment of the system in which the first metric and the second metric are collected by a medical toilet and transmitted electronically to a computer.

FIG. 5 is a perspective view of one embodiment of the system in which the first metric is an EKG reading and the second metric is collected by a medical toilet which then conducts a follow-up metric.

FIG. 6 is a flow chart illustrating a process through which the computer initiates a follow-up metric, receives the follow-up metric, and identifies a relevant physical variable in the user.

FIG. 7 is a perspective view of one embodiment of the system in which both a first metric and a second metric are collected by a medical toilet and a healthcare provider enters information about a user.

FIG. 8 is a flow chart illustrating a process through which information entered into the computer by a healthcare provider alters the first and second set of rules used to analyze a first metric.

### DETAILED DESCRIPTION OF THE INVENTION

Definitions

Toilet, as used herein, means a device that is configured to collect biological waste products of a mammal including urine and feces.

Medical toilet, as used herein, means a toilet that conducts one or more metrics relevant to a user's health status. This may include, but is not limited to, quantification of analytes in urine or feces as well as others, including cardiovascular parameters, bioimpedance measurements, and body weight.

Metric, as used herein, means a system, method, or standard of measurement.

Heath metric, as used herein, means a metric which measures a physiological characteristic or physiological function that is relevant to assessment of a user's health status.

Data, as used herein, means information, numerical or otherwise, that is collected using one or more of a variety of health metrics.

Health status, as used herein, means the current physiological state of a mammal, particularly with regard to disease status or injury. In general, this term refers to the overall health of the mammal. However, individual parameters relating to a specific body part or biological system may be measured for the purpose of diagnosing disease states or identifying physiological characteristics or functions that are outside of the normal range. Such individual physiological characteristics or functions may be used to define the health status of the mammal with regard to a specific physiological system.

User, as used herein, means any mammal, human or animal, for which the medical toilet disclosed herein is used to measure physiological functions which may be used to assess the mammal's health status.

Healthcare provider, as used herein, means any individual who performs a task, mental or physical, in relation to health-related services provided to a user. In addition to clinicians who practice medicine directly on a user, the term healthcare provider includes any person that enters data into a computer, when the data entry is used in analysis of a user's health status or to improve a user's health.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

Disclosed herein is a health metric validation system. A first metric that either directly indicates or infers a user's health status is collected. Normally, a clinician or other healthcare provider would interpret the data at this point with only a general knowledge about the inherent limitations of the health metric and no information about the validity of the health metric in this specific instance. However, according to the invention, a second metric is collected. The second metric may be known to provide an indication of the validity of the first metric. A first set of rules is then applied to the first and second metrics which assign a weight value to the first metric. The weight value is a function of the second metric. A second set of rules is applied to the weighted first metric to determine an indicator value. The indicator value is a function of the weight value and provides an indication of the validity of the first data set and, consequently, its relevance to a user's health status. The second set of rules may define a threshold value for the indicator value and may flag the first metric as invalid or to be excluded from multi-variable calculations that provide an assessment of the user's health status. A clinician may choose to interpret a first metric that has a mid-range indicator value in combination with more reliable health metrics to bolster the validity of a general trend shown by the first metric. Thus, the first metric provides some value but is not assigned more relevance than it merits. As one of skill in the art will understand, the combination of the first metric and the indicator value have a plurality of uses in assessing health metrics and their application to diagnostic efforts.

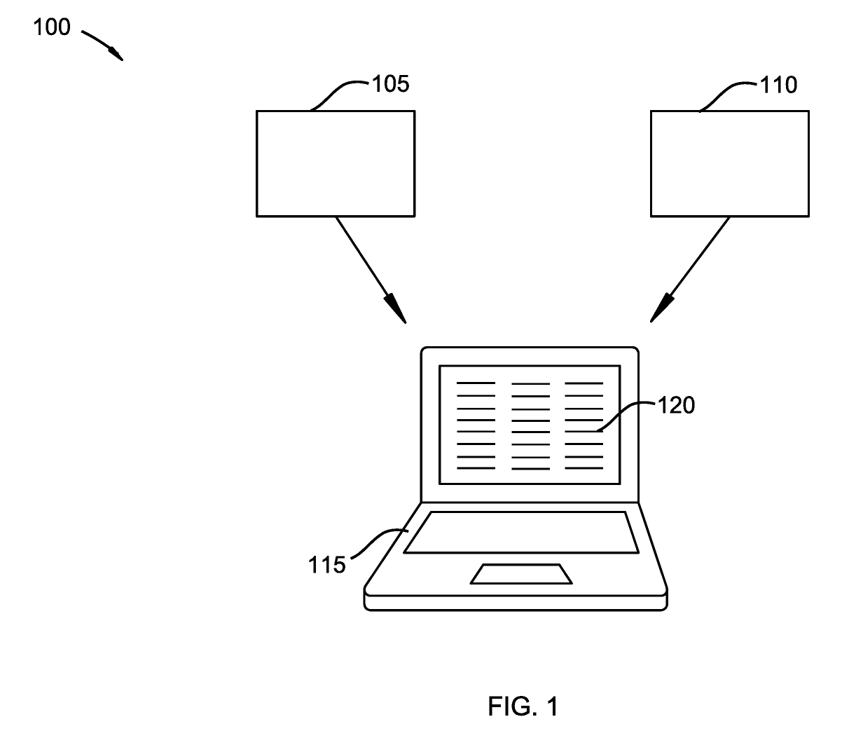

Referring now to the figures, FIG. 1 illustrates health metric validation system 100 which is an embodiment of the invention in which a first medical device 105 collects a first metric and a second medical device 110 collects a second metric. Through means that may include wireless transmission, an Ethernet, transfer through a flash drive, or direct manual entry, the first metric and second metric are entered into and stored on computer 115 which applies the rules and performs calculations as described herein. While schematically depicted as a laptop computer, computer 115 may be a server, a computer in a healthcare facility, or any other computing device that may receive and store data, be programed to perform calculations on the data, and provide an output of the calculated data. Accordingly, the screen of computer 115 is shown to present a report 120 of the first metric and other relevant information, including the indicator value of the first health metric.

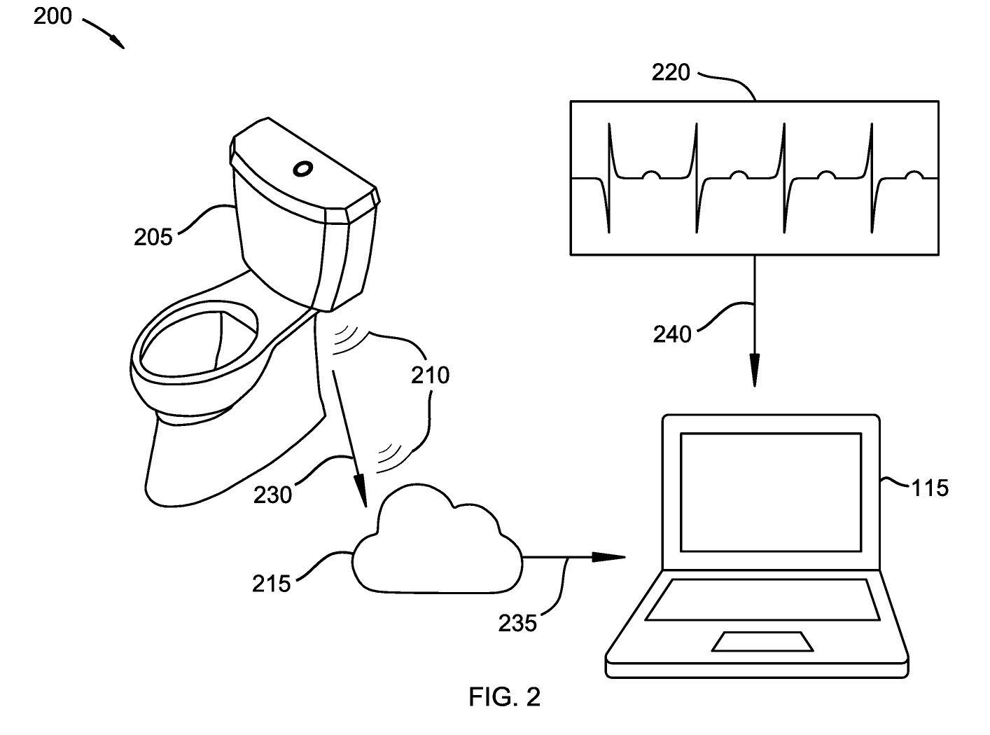

FIG. 2 illustrates health metric validation system 200 which is another embodiment of the invention. In this embodiment, a first metric is EKG reading 220 which is transmitted or otherwise entered into computer 115 through means 240. Means 240 may be wireless transmission, an Ethernet, transfer through a flash drive, or direct manual entry. The second metric is collected by medical toilet 205. The second metric is then transmitted through means 230, which, in this embodiment, comprises wireless signal 210. The second metric is transferred to network database 215, which may be the healthcare provider's server, via, for example, Cloud technology. The second metric is then downloaded to computer 115 through means 235. Computer 115 then applies the rules and performs calculations as described herein.

The second metrics that medical toilet 205 may measure include, but are not limited to, body temperature, body weight, body composition (i.e. percent body fat, intracellular and/or extracellular water), heart rate, pedal pulse rate, blood pressure, blood oxygen saturation, electrocardiogram measurement, urine constituents and parameters including urine color, glucose, urea, creatinine, specific gravity, urine protein, electrolytes, urine pH, osmolality, human chorionic gonadotropin (for detecting pregnancy), hemoglobin, white blood cells, red blood cells, ketone bodies, bilirubin, urobilinogen, free catecholamines, free cortisol, phenylalanine, and urine volume. The metrics may also include fecal analysis including fecal weight and volume, calprotectin, lactoferrin, and hemoglobin. Additionally, the flow or volume or weight sensor may determine periods of excretion activity to measure, for instance, urination or defecation exertions, or selectively record metrics that were collected during periods of low exertion. These metrics are useful because some metrics are best performed after complete voiding of the bowel and bladder for maximum accuracy.

In addition, multiple second metrics may be collected and used as described herein to assess the validity of the first metric. Alternatively, a single second metric may be used to assess the validity of multiple metrics that comprise the first metric in the disclosed health metric validation system.

As one of skill in the art will readily understand, the first metric may comprise of metrics other than an EKG reading. In alternative embodiments, the first metric may be any of those listed above as metrics that may be collected by medical toilet 205. In other embodiments, the first metric may comprise of any of stress test, blood pressure, hematocrit, serum insulin level, hemoglobin A1c, breathing rate, blood urea nitrogen, serum creatinine, alanine am inotransferase, aspartate am inotransferase, alkaline phosphatase, serum bilirubin, serum total protein, serum albumin, serum gamma-glutamyl transpeptidase, prothrombin time, Holter monitoring, serum levels of a pharmaceutical product, and serum levels of a metabolite of a pharmaceutical product.

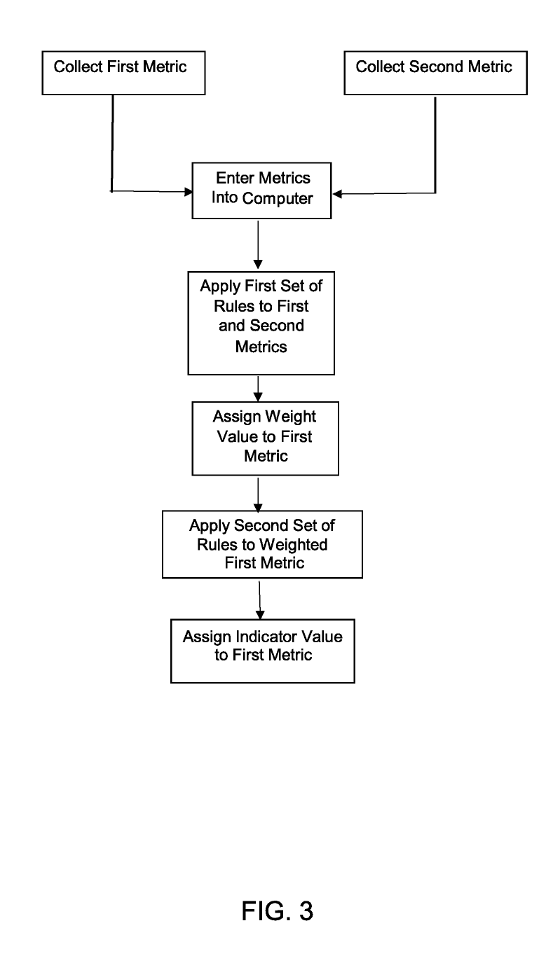

FIG. 3 is a flow chart illustrating an embodiment of the health metric validation system which may be either of those illustrated in FIGS. 1 and 2. In the illustrated process, a first metric and a second metric are collected. Either or both of these metrics may be collected by at least one medical device, including, but not limited to, one or more of a medical toilet, a physical exam performed by a clinician, or a laboratory assay of an analyte. In this embodiment, both the first and second metrics are entered into a computer. This step may occur by manual data entry or a variety electronic transmission methods known in the art. The computer is programmed to perform calculations and apply a first set of rules to the first and second metrics. The computer applies the first set of rules then calculates and assigns a weight value to the first metric. The weight value is a function of the second metric. The computer then applies a second set of rules to the weighted first metric. The computer assigns an indicator value to the first metric. The indicator value is a function of the weight value. The indicator value provides information about the validity of the first metric. A healthcare provider may use this indicator value to make a decision about the use of the first metric. For example, the healthcare provider may decide whether to use the first metric as an indicator of a user's health status, use the first metric but only interpret it in combination with more valid metrics that bolster the implication of the first metric, ignore the first metric and recollect it, perhaps under more optimal conditions, or conduct an alternative metric.

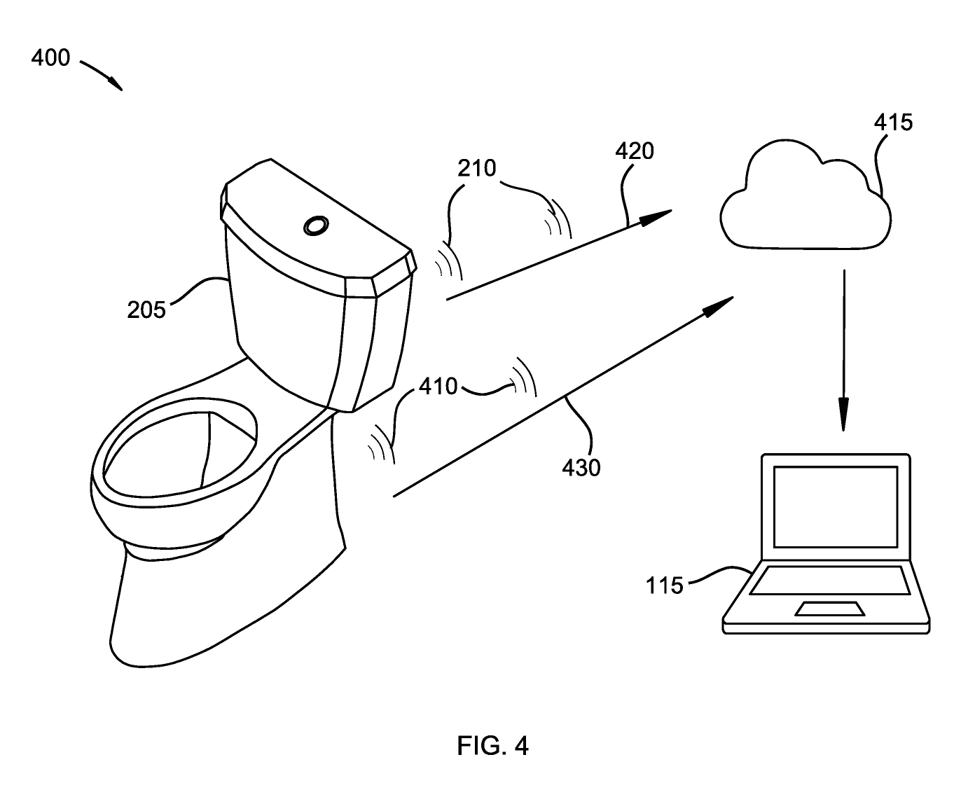

FIG. 4 illustrates health metric validation system 400, which is yet another embodiment of the disclosed invention. In this embodiment, both the first metric and the second metric are collected by medical toilet 205. The wireless signal 210 transmits the first metric through means 420 and wireless signal 420 transmits the second metric through means 410. Both the first metric and the second metric are transmitted to network database 215 which then downloads the first and second metrics onto computer 115. Computer 115 then applies the rules and performs calculations as described herein.

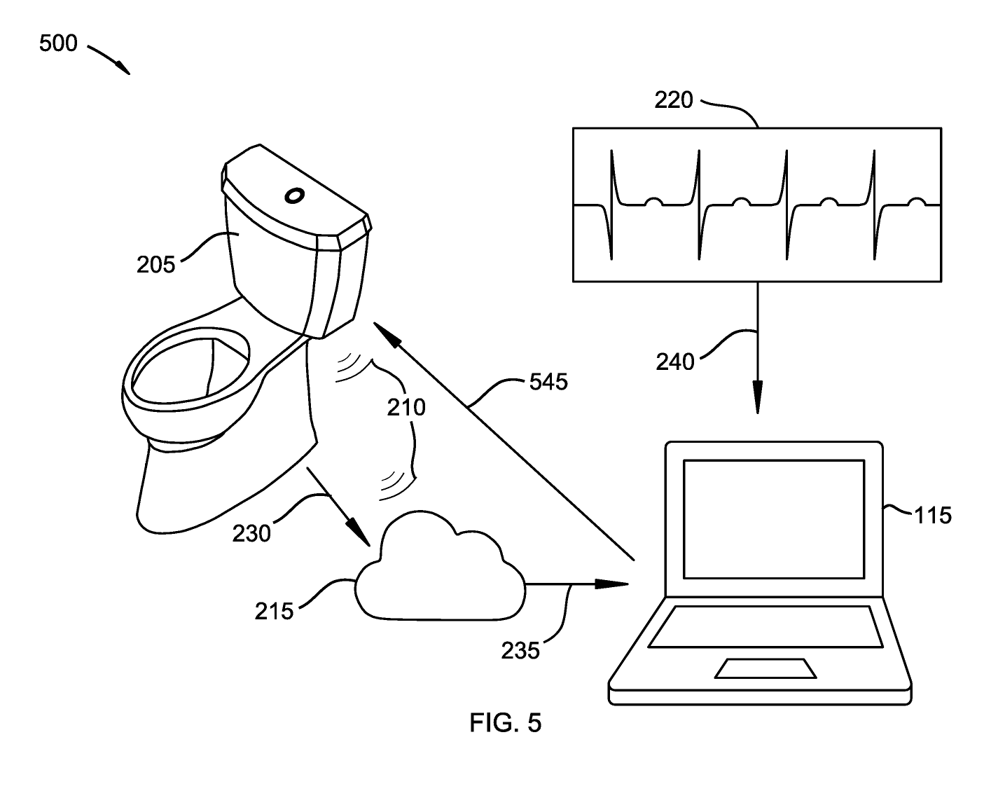

FIG. 5 illustrates health metric validation system 500, which is yet another embodiment of the disclosed invention. In this embodiment, the first metric is EKG reading 220 which is transmitted or otherwise entered into computer 115 through means 240. Means 240 may be wireless transmission, an Ethernet, transfer through a flash drive, or direct manual entry. The second metric is collected by medical toilet 205. The second metric is then transmitted through means 230, which, in this embodiment, comprises wireless signal 210. Second metric is transferred to network database 235, which may be the healthcare provider's server, via, for example, Cloud technology. The second metric is then downloaded to computer 115 through means 235. Computer 115 then applies the rules and performs calculations as described herein. Up to this point, health metric validation system 500 is similar to the embodiment of FIG. 2. However, in this embodiment, calculations performed on computer 115 have determined that a follow-up metric is needed. The reasons a follow-up metric may be needed include a poor indicator value assignment to the first metric. A poor indicator value may mean that the first metric was not collected under optimal conditions and, therefore, resulted in a poor reading. Alternatively, the user may possess a specific physiological characteristic that suggests that further information about the user is needed to properly interpret the first variable. Physiological characteristics that may indicate a need for a follow-up metric include, but are not limited to body type, gender, skeletal structure (fine or heavy) and whether or not the user is afflicted with a certain disease.

For example, if the individual's height is entered into computer 115, a body weight measurement may be used to calculate a body mass index (BMI) which is weight expressed in kilograms divided by height squared in meters (BMI=Weight/(Height)²). An extremely healthy and fit athlete with a low percent body fat may have a high BMI and erroneously be interpreted to be unhealthy. But, a follow-up metric comprising a bioimpedance measurement may be used to determine the user's percent body fat. If this follow-up metric suggests that the user does indeed have a low percent body fat, an alternative second set of rules may be applied to the first metric. In this situation, the report provided by computer 115 may indicate that the BMI is accurate, but not valid because the user has a body type for which BMI is not a useful indicator of health status.

Other physiological characteristics that may suggest that a follow-up metric would assist in interpreting the first metric include metrics which identify dehydration, hypervolemia, hypovolemia; pregnancy, electrolyte imbalance, the presence of a metabolite of a food that interferes with the accurate measurement of the first metric, and the presence of a pharmaceutical product or a metabolite thereof, when the pharmaceutical product or its metabolite interferes with the accurate measurement of the first metric.

For example, the first metric may be a cardiovascular indicator such as heart rate or blood pressure. If the first metric is outside of normal range, the data suggest that the user has a compromised overall health status. However, a follow-up metric that comprises an analysis of the same individual's urine may indicate dehydration. In this scenario, the abnormal heart rate or blood pressure are likely to be temporary. The follow-up metric may trigger the application of an alternative second set of rules to the first metric. The report provided by computer 115 after applying the alternative second set of rules may indicate that the heart rate or blood pressure measurement is accurate, but not valid because the user is dehydrated. A set of measurements taken at another time, this time when the individual is properly hydrated, may then be used to give a more accurate health status assessment.

In another example, a first metric may be a heart rate measurement taken by a medical toilet through a stethoscope positioned on the tank of the medical toilet. A user that is seated on the toilet leans back against the stethoscope to begin collection of the metric. However, if the user is wearing heavy clothing or not leaning squarely against the stethoscope, a valid heart rate metric may not be collected. A second metric may comprise of a temperature sensor that may be positioned near the stethoscope. The temperature detected by the temperature sensor may provide an indication of whether stethoscope is directly against the user's skin. If the measured temperature is significantly below normal body temperature, the indicator value for the heart rate metric may suggest poor validity. A follow-up metric that does not rely on the user's skin coming in contact with the stethoscope may provide more a more valid indicator of the user's health status. For example, a follow-up metric may comprise of an alternative method of measuring heart rate such as bioimpedance measurements.

In addition, the follow-up measurement may be accompanied by a third metric which may be used to assess the validity of the follow-up metric. In this embodiment, the process for evaluating the follow-up metric is similar or identical to that of the first measurement except that the first and second sets of rules are applied to follow-up metric and third metric as if they were the first metric and the second metric. A weight value and indicator value are assigned to the follow-up metric as they were for the first metric. This process may be repeated until a valid metric is acquired.

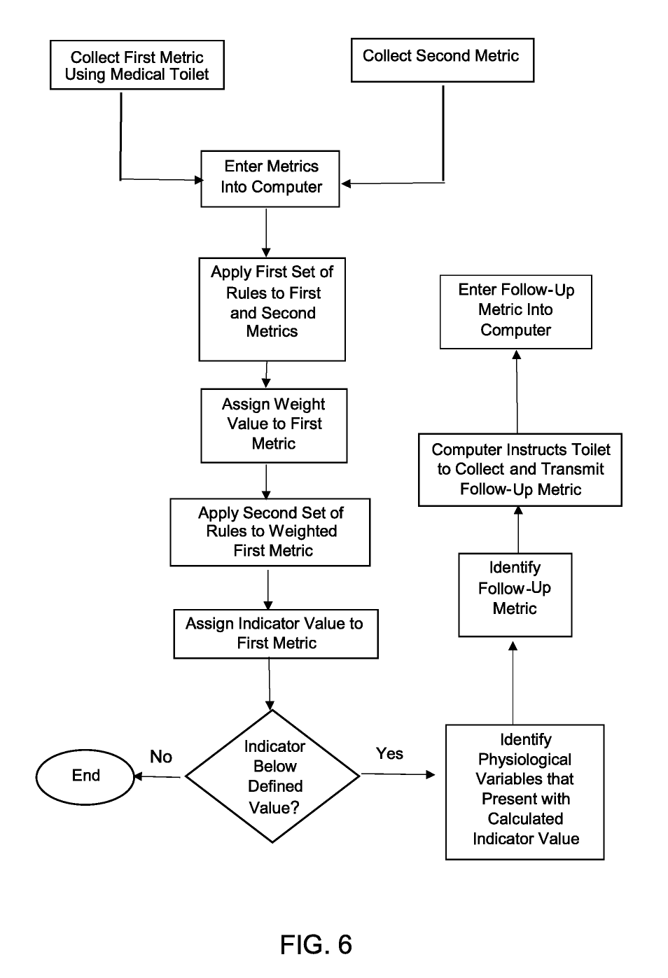

FIG. 6 is a flow chart illustrating the use of follow-up metrics to provide an accurate measurement of a specific physiological characteristic or function in a user. In this embodiment, at least one of the first metric and the second metric is presumed to be collected from a medical toilet although other methods of data collection may be used in other embodiments. A first metric and a second metric are collected and entered into a computer. A first set of rules is applied to the first and second metric. The calculations performed by applying the first set of rules produces a weight value which is assigned to the first metric. A second set of rules is applied to the weighted first metric and an indicator value is assigned to the first metric. If the indicator value is below a defined value, the computer may send a signal to the medical toilet triggering a follow-up metric. The follow-up metric may be an alternative method to assess the physiological characteristic or function that the first metric attempted to measure. A third metric is also collected to assess the validity of the follow-up metric. The first and second sets of rules are applied to the follow-up metric and the third metric just as they were for the first and second metrics. If the follow-up metric is assigned an indicator value above a defined level, the process ends. If not, the process may repeat until a valid metric is acquired.

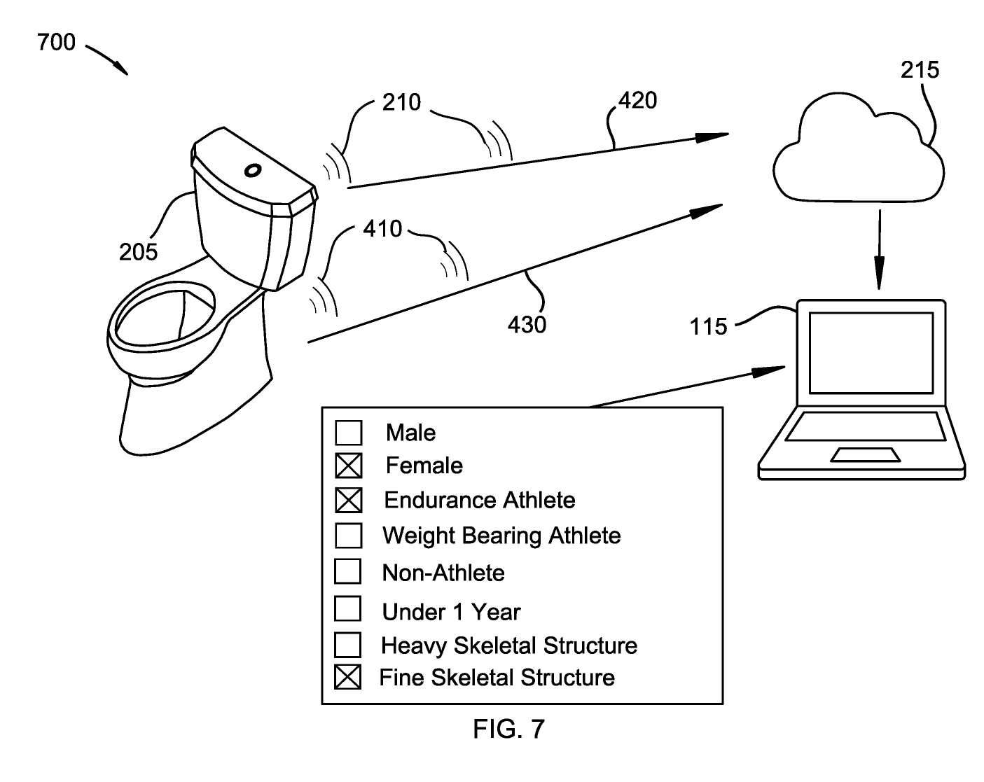

FIG. 7 illustrates health metric validation system 700, which is yet another embodiment of the disclosed invention. In this embodiment, a healthcare provider enters data about the user's physiological characteristics into computer 115. The data may be relevant to interpretation of the first metric. In this embodiment, medical toilet 205 collects both the first metric and the second metric although other methods of metric collection may be used in other embodiments. As described with reference to FIG. 5, different physiological characteristics associated with a user may impact the most accurate and meaningful interpretation of the first metric. By providing this information about the user, a follow-up metric to assess whether or not the user has a relevant characteristic is not needed. The computer will apply the appropriate set of rules during the first calculation and provide a report that references the implication of the first metric with regard to the user's health status in view of the relevant physiological characteristic.

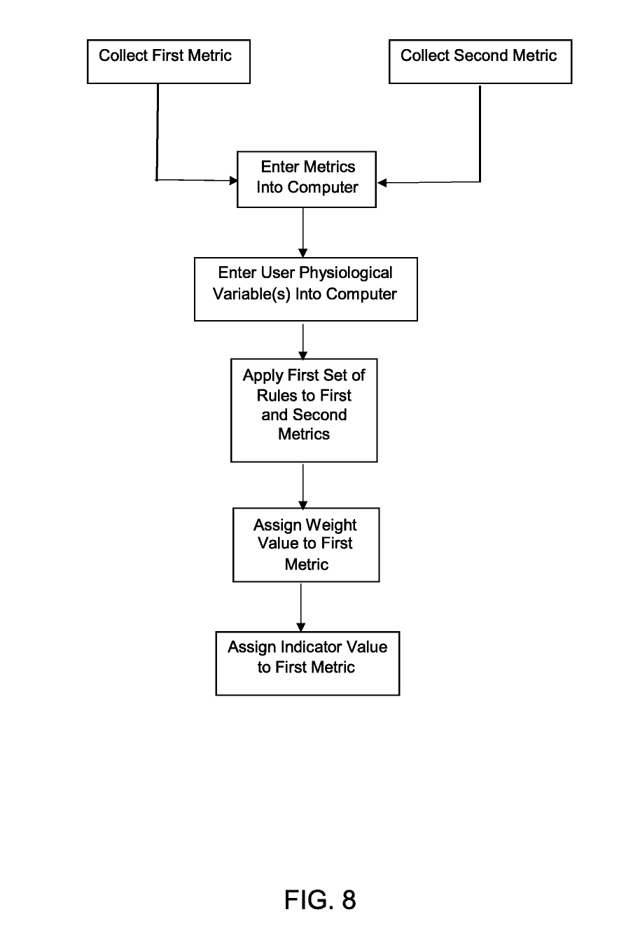

FIG. 8 is a flow chart which illustrates the use of health metric validation system 700. A first metric and a second metric are collected and the data entered into a computer. A healthcare provider enters information about the user's physiological characteristics into the computer. As one of skill in the art will understand that the user's physiological characteristics may be entered into the computer through methods other than manual data entry. For example, the computer may be programmed to obtain information about the user's physiological characteristics electronically by copying the information from a specific field in the user's electronic medical record file stored in a database.

The first set of rules is applied to the first and second metrics. A weight value is assigned to the first metric. A second set of rules is applied to the weighted first metric and an indicator value is assigned to the weighted first metric. In this embodiment, the first and second sets of rules are those that are appropriate for processing the metrics according to the information about the user's physiological characteristic(s).

Both the first set of rules and the second set of rules may vary with each type of metric. This is because rules that are specifically relevant to the particular metric may be included in the sets.

Examples of parameters which may be addressed in the first set of rules may include consistency of first metric signal, strength of first metric signal, consistency of first metric signal relative to consistency of second metric signal, strength of first metric signal relative to strength of second metric signal, presence or absence of related analyte(s) in second metric, quantitative amount of related analyte(s) in second metric, presence or absence of a defined and measurable second metric signal, and a minimum or maximum value of a quantitative signal measured by a second metric.

Examples of parameters which may be addressed in the second set of rules may include whether the weight value is above a threshold defined for the first metric, whether the weight value is within a medium range defined for the first metric, whether the weight value is within a high range defined for the first metric, and whether the weight value indicates a need for a follow up metric.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A health metric validation system, comprising:
a computer;
wherein the computer comprises a non-transitory computer readable medium that instructs the computer to receive and store a first health metric and a health second metric,
wherein the first metric and the second metric comprise of measurements performed on a user by at least one medical device;
wherein the first metric is an indicator of a user's health status;
wherein the second metric is an indicator of the validity of the first metric;
wherein the non-transitory computer readable medium assigns a weight value to the first metric, the weight value being:
determined according to a first set of rules,
a function of the second metric, and
proportional to the validity of the first metric; and
wherein the non-transitory computer readable medium further instructs the computer to assign an indicator value to the first metric according to a second set of rules, wherein the indicator value is proportional to the validity of the first metric.

2. The health metric validation system of claim 1, wherein the first metric is collected using a medical toilet.

3. The health metric validation system of claim 1, wherein the second metric is collected using a medical toilet.

4. The health metric validation system of claim 3, wherein the first metric is collected using a medical toilet.

5. The health metric validation system of claim 3, wherein the toilet comprises a device for measuring one or more properties of a user's biological waste.

6. The health metric validation system of claim 5, wherein the at least one property of the user's biological waste is selected from one or more of the following:
urine color, urine glucose concentration, urine urea concentration, urine creatinine concentration, urine specific gravity, urine protein concentration, urine electrolyte concentrations, urine pH, urine osmolality, urine human chorionic gonadotropin concentration, urine hemoglobin level, white blood cells in urine, red blood cells in urine, urine ketone body concentration, urine bilirubin concentration, urine urobilinogen concentration, urine free catecholamine concentration, urine free cortisol concentration, urine phenylalanine concentration, urine volume, fecal volume, fecal weight, fecal calprotectin level, fecal lactoferrin level, fecal hemoglobin level, urine levels of a pharmaceutical compound, urine levels of a metabolite of a pharmaceutical compound, fecal levels of a pharmaceutical compound, and fecal levels of a metabolite of a pharmaceutical compound.

7. The health metric validation system of claim 3, wherein the first metric is selected from one or more of the following:
electrocardiogram analysis, heart rate, stress test, blood pressure, hematocrit, serum insulin level, hemoglobin A1c, breathing rate, blood urea nitrogen, serum creatinine, alanine am inotransferase, aspartate am inotransferase, alkaline phosphatase, serum bilirubin, serum total protein, serum albumin, serum gamma-glutamyl transpeptidase, prothrombin time, Holter monitoring, serum levels of a pharmaceutical product, and serum levels of a metabolite of a pharmaceutical product.

8. The health metric validation system of claim 3, wherein non-transitory computer readable medium instructs the computer to generate a first report.

9. The health metric validation system of claim 8, wherein the first report comprises:
the values of the first metric;
the indicator value assigned to the first metric; and
a list, the list comprising one or more physiological characteristics which are associated with misleading values for the first metric.

10. The health metric validation system of claim 9, wherein the first report further lists follow-up health metrics for use in identifying the presence of the one or more physiological characteristics.

11. The health metric validation system of claim 9, wherein the list comprises one or more of the following:
dehydration;
hypervolemia;
hypovolemia;
pregnancy;
electrolyte imbalance;
presence of a metabolite of a food, wherein the food interferes with the accurate measurement of the first metric;
the presence of a metabolite of a pharmaceutical product, wherein the pharmaceutical product interferes with the accurate measurement of the first metric; and
the presence of a pharmaceutical product, wherein the pharmaceutical product interferes with the accurate measurement of the first metric.

12. The health metric validation system of claim 1, wherein the medical toilet electronically transmits the first metric and the second metric to the computer.

13. The health metric validation system of claim 2, wherein the toilet transmits the first metric value to the computer through an electronic signal.

14. The health metric validation system of claim 3, wherein the medical toilet transmits the first metric value to the computer through an electronic signal.

15. The health metric validation system of claim 10, wherein the non-transitory computer readable medium initiates an electronic signal, and wherein the electronic signal instructs the medical toilet to collect at least one follow-up health metric.

16. The health metric validation system of claim 15, wherein the medical toilet sends an electronic signal to the computer, wherein the electronic signal transmits the at least one follow-up health metric to the computer.

17. The health metric validation system of claim 16, wherein non-transitory computer readable medium generates a second report, the second report comprising one or more of the following:
an indication of which of the one or more physiological characteristics has been excluded by the follow-up health metric; and
an indication of which of the one or more physiological characteristics has been confirmed by the follow-up health metric.

18. The health metric validation system of claim 9, wherein the one or more physiological characteristics comprises a plurality of different body types.

19. The health metric validation system of claim 18, wherein the plurality of different body types comprises of one or more of the following: endurance athlete, weight bearing athlete, non-athlete, male, female, under a defined age, over a defined age, fine skeletal structure, and heavy skeletal structure.

20. The health metric validation system of claim 1, wherein one or more of the first set of rules and the second set of rules varies according to the body type, and wherein a healthcare indicates the user's body type through an input mechanism provided through the non-transitory computer readable medium.
